(19) United States
(12) Patent Application Publication
(10) Pub. No.: US 2023/0347029 A1
(43) Pub. Date: Nov. 2, 2023

(54) DIALYSIS BIOMARKER MONITORING

(71) Applicant: ROCKLEY PHOTONICS LIMITED, Altrincham (GB)

(72) Inventors: Jennifer Lynn CORSO, Peoria, AZ (US); Craig GARDNER, Belmont, MA (US)

(21) Appl. No.: 18/310,487

(22) Filed: May 1, 2023

Related U.S. Application Data
(60) Provisional application No. 63/337,525, filed on May 2, 2022.

(51) Int. Cl. A61M 1/16; A61B 5/00; A61B 5/024

(52) U.S. Cl. CPC A61M 1/1601; A61B 5/0075; A61B 5/02416

(57) ABSTRACT

Dialysis patients may be affected by renal failure and may be affected by other health conditions, such as hypertension. During and between dialysis sessions, it may be advantageous to monitor various characteristics of the patient and of the dialysis system. As such, a system and method for dialysis biomarker monitoring is provided.

### CROSS-REFERENCE TO RELATED APPLICATION(S)

The present application claims priority to and the benefit of U.S. Provisional Application No. 63/337,525, filed May 2, 2022, entitled “DIALYSIS MONITORING SYSTEM WITH WEARABLE DEVICE”, the entire content of which is incorporated herein by reference.

The present application is related to U.S. Pat. Application Ser. No. 17/757,130, filed on Jun. 9, 2022, entitled “OPTICAL SENSING MODULE”, the entire content of which is incorporated herein by reference.

### FIELD

One or more aspects of embodiments according to the present disclosure relate to sensing, and more particularly to sensing of biomarkers in the context of dialysis.

### BACKGROUND

Dialysis patients may be affected by renal failure and may be affected by other health conditions, such as hypertension. During and between dialysis sessions, it may be advantageous to monitor various characteristics of the patient and of the dialysis system.

It is with respect to this general technical environment that aspects of the present disclosure are related.

### SUMMARY

According to an embodiment of the present disclosure, there is provided a system, including: a housing, sized and dimensioned to be disposed about the body of a patient; and a spectrophotometer, in the housing, the spectrophotometer being configured to illuminate the skin of the patient with probe light and to perform measurements of return light, the return light being light returning, to a photodetector of the spectrophotometer, from the skin of the patient, as a result of the illumination by the probe light, the spectrophotometer having an operating wavelength range including a wavelength between 2080 nm and 2400 nm.

In some embodiments, the spectrophotometer is configured to measure urea nitrogen.

In some embodiments, the housing has a volume of less than 100 cubic centimeters.

In some embodiments, the system further includes a radio in the housing, wherein the system is configured to transmit data, via the radio, the data being based on the measurements.

In some embodiments, the transmitting of the data includes transmitting the data to a dialysis system.

In some embodiments, the transmitting of the data to the dialysis system includes transmitting the data to the dialysis system wirelessly.

In some embodiments, the transmitting of the data to the dialysis system includes transmitting the data during a dialysis session, the data being based on measurements performed during the dialysis session.

In some embodiments, the transmitting of the data to the dialysis system includes transmitting the data during a dialysis session, the data being based on measurements performed before the dialysis session.

In some embodiments, the system further includes a photoplethysmography (PPG) sensor and a speckle plethysmography (SPG) sensor.

In some embodiments, the measurements are suitable for estimating a quantity selected from the group consisting of heart rate, blood pressure, blood flow, heart rate variability, hemoglobin, blood oxygen saturation, and respiratory rate.

In some embodiments, the measurements are suitable for estimating a concentration of a biomarker in tissue or fluid of the patient.

In some embodiments, the biomarker is selected from the group consisting of water, glucose, hemoglobin, creatinine, urea, lactate, ethanol, and albumin.

In some embodiments, the system further includes a band configured to secure the housing against the skin of the patient.

In some embodiments, the band is configured to secure the housing against the skin of the wrist of the patient.

In some embodiments, the system further includes an adhesive patch configured to secure the housing against the skin of the patient.

According to an embodiment of the present disclosure, there is provided a method, including: performing a measurement with a spectrophotometer, and transmitting the measurement to a dialysis system, the performing of the measurement including: illuminating the skin of a patient with probe light; and performing measurements of return light, returning, to a photodetector of the spectrophotometer, from the skin of the patient, as a result of the illumination by the probe light.

In some embodiments, the transmitting of the measurement to the dialysis system includes transmitting the measurement directly to the dialysis system.

In some embodiments, the transmitting of the measurement to the dialysis system includes transmitting the measurement to the dialysis system via the internet.

In some embodiments, the transmitting of the measurement to the dialysis system includes transmitting the measurement during a dialysis session, the measurement being a measurement performed during the dialysis session.

In some embodiments, the transmitting of the measurement to the dialysis system includes transmitting the measurement during a dialysis session, the measurement being a measurement performed before the dialysis session.

### BRIEF DESCRIPTION OF THE DRAWINGS

These and other features and advantages of the present disclosure will be appreciated and understood with reference to the specification, claims, and appended drawings wherein:

FIG. 1A is a block diagram of a dialysis system, according to an embodiment of the present disclosure;

FIG. 1B is a schematic illustration of a sample holder arrangement, according to an embodiment of the present disclosure;

FIG. 1C is a schematic illustration of a sample holder cartridge, according to an embodiment of the present disclosure;

FIG. 1D is a schematic illustration of a conduit with connectors, according to an embodiment of the present disclosure;

FIG. 1E is a schematic illustration of a sample holder arrangement, according to an embodiment of the present disclosure;

FIG. 1F is a schematic illustration of a sample holder arrangement, according to an embodiment of the present disclosure;

FIG. 2A is a block diagram of a biomarker sensor, according to an embodiment of the present disclosure;

FIG. 2B is a block diagram of a biomarker sensor, according to an embodiment of the present disclosure;

FIG. 2C is a block diagram of a biomarker sensor, according to an embodiment of the present disclosure;

FIG. 2D is a block diagram of a biomarker sensor, according to an embodiment of the present disclosure;

FIG. 3A is a block diagram of a controllable dialysate source, according to an embodiment of the present disclosure;

FIG. 3B is a block diagram of a controllable dialyzer, according to an embodiment of the present disclosure;

FIG. 4 is a block diagram of a dermal sensor and associated components, according to an embodiment of the present disclosure;

FIG. 5A is a flow chart of a use case, according to an embodiment of the present disclosure; and

FIG. 5B is a flow chart of a use case, according to an embodiment of the present disclosure.

### DETAILED DESCRIPTION

The detailed description set forth below in connection with the appended drawings is intended as a description of exemplary embodiments of a system and method for monitoring during and between dialysis sessions provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

Renal replacement therapy (RRT) is defined as a replacement for native kidney filtration for those with acute and chronic renal failure, as well as some instances of acute kidney injury. Dialysis and kidney transplants are both types of RRT. Dialysis acts as an artificial filter, removing unwanted substances from circulation, such as uremic toxins, electrolytes, and other metabolites through either peritoneal or hemofiltration. Dialysis may be implemented when native kidney function falls below 15% and the estimated glomerular filtration rate (eGFR) is less than 30 mL/min. Dialysis may also be used to treat toxicity (e.g., alcohol poisoning), in which case it may be used to remove the toxin from the patient’s blood, or during acute kidney injury when warranted.

Dialysis employs a solution (dialysate) with known solute concentrations, a semipermeable membrane, and either the mode of passive diffusion or convective clearance to filter the blood of a patient. Dialysis patients may spend anywhere from 9 to 24 hours per week on dialysis, with doses lasting, e.g., from 2-8 hours; some patients may receive continuous dialysis 24 hours per day. Both dialysis dosing and the type of dialysis may depend on the severity of disease, overall health status, presence and type of comorbidities, and risk of complications.

Some patients begin dialysis while waiting on a donor kidney to become available. In comparison to dialysis, evidence supports a higher quality of life and survivability once a kidney transplant is received. Advancing age and time spent on dialysis both increase the risk of transplant complications and failure. There may be a need for improvements in dialysis dosing through real-time monitoring of biomarkers associated with typical adequacy assessments during treatment, and between treatments. This may include monitoring a patient’s urea, and additional aspects important in indicating a patient’s status, including blood pressure and other cardiovascular responses to treatment such as blood flow and clotting factors, fluid levels and balance, hemoglobin concentration, temperature stability, glucose levels, and changes in creatinine and albumin.

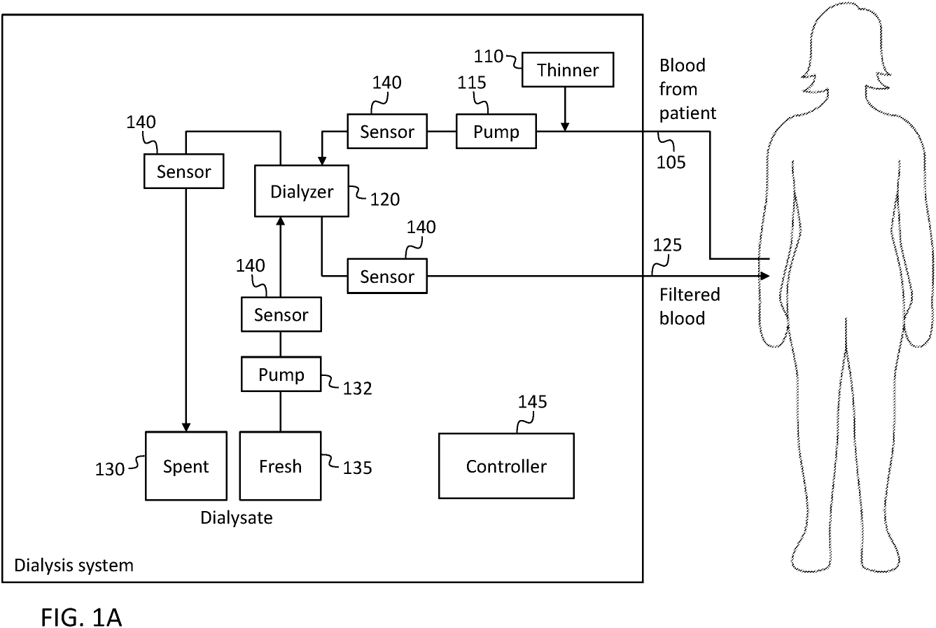

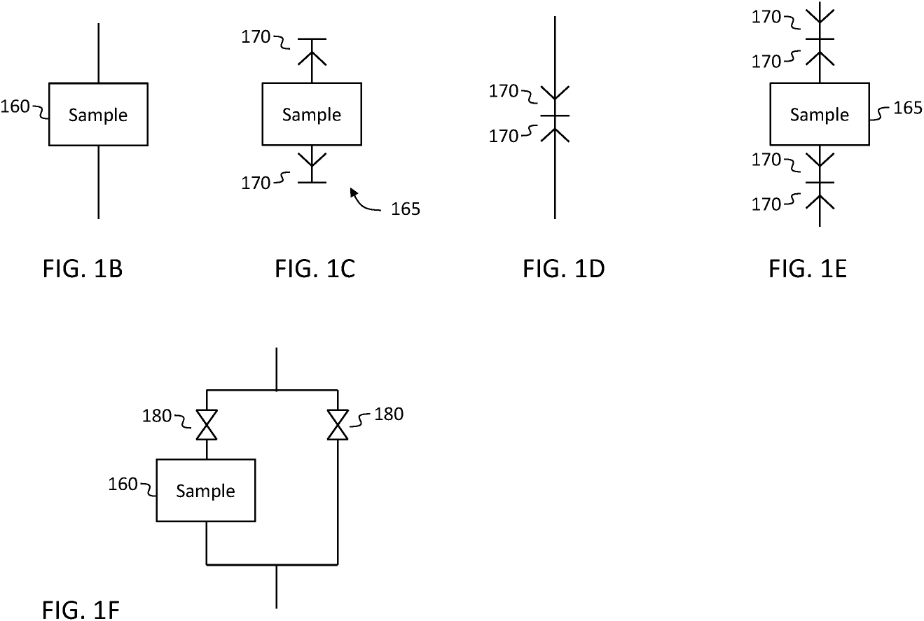

Dialysis may be performed by filtering toxins and waste solutes from either the blood or peritoneum. Access to the patient’s blood may be done through a surgically-created arteriovenous fistula. FIG. 1 shows a dialysis system (for performing hemodialysis), in some embodiments. Blood from a patient leaves the body via a tube in the arm, is received, by the dialysis system, at an input 105; blood thinner from a thinner source 110 is added to the flow of blood, which is pumped by a blood pump 115 (e.g., a peristaltic pump) through a dialyzer 120, to produce filtered blood, which is returned to the arm of the patient via an output 125 of the dialysis system.

The dialyzer 120 allows water, urea, creatinine and other small molecules to pass through the filter, but prevents larger protein molecules such as hemoglobin and albumin from leaving the blood. Counter-flowing to the blood in the dialyzer is the dialysate fluid. Dialysate, from a dialysate source 130 (e.g., a dialysate reservoir), passes through (e.g., is pumped, by a dialysate pump 132 (which may be a peristaltic pump sharing an actuator with the blood pump 115), through) the dialyzer 120 and then returns to a spent dialysate reservoir 135. Due to both a pressure gradient and a concentration gradient, water, urea and other small molecules pass into the dialysate through the membrane of the dialyzer 120 and therefore do not return to the body. A controller 145 (which may be or include a processing circuit) may monitor and control the operation of the dialysis system.

Evidence supports that removing a certain percentage of urea may help patients with kidney failure live longer; however, removing too much water along with urea can cause short-term complications during and immediately after treatment, such as low blood pressure, nausea, or fainting. Therefore, a method to monitor the amount of urea removed in real time may be advantageous. Clinicians target a urea reduction ratio (URR) greater than 65%, where the URR is a measure of the reduction in blood urea nitrogen (BUN). The urea reduction ratio may be defined to be the ratio of (i) the change in the urea level in the blood during a dialysis session to (ii) the urea level in the blood at the beginning of the dialysis session.

To provide such real-time monitoring, one or more biomarker sensors 140 may be affixed to one or more respective fluid conduits in the dialysis system and the fluid in the conduits may be monitored to measure (or estimate) the concentrations of various biomarkers. As used herein, a “biomarker” is a quantity of clinical significance that can be measured, such as (i) a chemical that may be present in biological tissue or fluid, the concentration of which is of interest, or (ii) a vital sign (such as the heart rate or blood pressure) of a patient. Each biomarker sensor 140 may measure the concentration of one or more biomarkers in the fluid in the conduit to which the biomarker sensor 140 is affixed. In some embodiments, each biomarker sensor 140 includes a spectrophotometer. As discussed in further detail below, the spectrophotometer may illuminate the sample (e.g., the volume of fluid being characterized by the spectrophotometer) with probe light of various different wavelengths, and it may measure the return light, which is light that returns to a photodetector in the spectrophotometer, from the sample, as a result of the illumination by the probe light. From such measurements it may be possible to infer the concentrations of various biomarkers in the sample, as discussed in further detail below. The biomarkers measured may include water, glucose, albumin, D-dimer, hemoglobin, creatinine, urea, lactate, and potential toxins removed using dialysis including ethanol, methanol, valproate and theophilline. Each biomarker sensor 140 may perform measurements periodically, e.g., once per minute (or with a frequency between once per hour and 10,000 times per hour). Additional sensors may be employed during a dialysis session; for example, the patient may wear an ambulatory blood-pressure cuff making it possible to monitor blood pressure in real time.

In some embodiments, a biomarker sensor 140 may be affixed (by a suitable clip) to a section of transparent tubing (e.g., perfluoroalkoxy (PFA) tubing, or any other tubing that is (i) sufficiently transparent over the operating wavelength range of the biomarker sensor 140 and (ii) biocompatible) and the sample volume is the volume of fluid, in the section of tubing, that is illuminated by the probe light and from which light is able to return to the photodetector. The optical path through the fluid may be about 0.5 mm; this may be accomplished, if the inner diameter of the tubing exceeds the desired optical path length, by squeezing the tube so that its cross section is oblong. In other embodiments, the sample volume is the interior of a sample holder constructed to have (i) a fluid passage and (ii) optical characteristics that are favorable for the transmission of the probe light into the fluid in the fluid volume and the return of the return light to the photodetector. For example, the sample holder may be composed of glass, and it may have a first polished surface facing the spectrophotometer, or a second polished surface in contact with the fluid, or both. In such an embodiment, to reduce loss due to reflections, one or both of the polished surfaces may have an antireflection coating, or a transparent index-matching compound may be used to fill the gap between the output aperture of the spectrophotometer and the first polished surface. One or more antireflection coatings or a layer of transparent index-matching compound may similarly be used to reduce optical loss between the fluid and the photodetector of the spectrophotometer.

The sample holder 160 (whether it is a section of tubing or, for example, a glass sample holder) may be constructed to be a permanent part of a fluid conduit of the dialysis system, as shown in FIG. 1B, or it may be a cartridge 165 having a connector 170 (e.g., a quick-connect connector) at each end, as shown in FIG. 1C. If a cartridge is used, the fluid conduit may have a pair of in-line connectors 170, so that fluid may flow through the conduit when the cartridge has not been installed (FIG. 1D) and so that the cartridge may be connected in line with the fluid conduit as shown in FIG. 1E. In some cases, a bypass path may be present, as illustrated in FIG. 1F. In the embodiment of FIG. 1F, a first valve 180 is present in the path with the sample holder 160 and a second valve 180 is present in the bypass path. Each of the valves 180 may be electrically actuated (and, e.g., controlled by the controller 145). Closing the valve 180 in the bypass path and opening the valve in the path with the sample holder 160 may cause the fluid to flow through the sample holder. Subsequently opening the valve 180 in the bypass path and closing the valve in the path with the sample holder 160 may cause the fluid to flow instead through the bypass path, causing the fluid in the path with the sample holder 160 to become stationary. Such an arrangement may be advantageous if fluid motion degrades the quality of the measurements of the biomarker sensor 140.

In embodiments in which the sample holder 160 is constructed to be a permanent part of a fluid conduit of the dialysis system, as shown in FIG. 1B, the combination of the dialyzer 120, the fluid conduit for blood from the patient (along with the sample holder 160, if any, integrated into this conduit) the fluid conduit for filtered blood (along with the sample holder 160, if any, integrated into this conduit), and the fluid conduits for dialysate (along with the sample holders 160, if any, integrated into these conduits) may be one single-use (e.g., disposable) component of the system, that may be replaced for each dialysis session. In some embodiments, each of the biomarker sensors 140 is integrated with its respective sample holder 160 and is also part of this single-use component. In embodiments in which the sample holder 160 is a cartridge, the dialyzer 120 and the conduits (some or all of which may be equipped with in-line pairs of connectors 170) may be a single-use component, and the sample holder cartridges 165 (or cartridges 165 with integrated biomarker sensors 140) may be separate single-use components to be installed as needed (e.g., depending on the monitoring requirements of the patient). Each of the biomarker sensors 140 may be connected to a power supply of the dialysis system, and may receive power (e.g., DC electrical power) from the dialysis system (which may be connected to wall power). In some embodiments, a heater or cooler (e.g., a Peltier cooler) may be used to control the temperature of the sample.

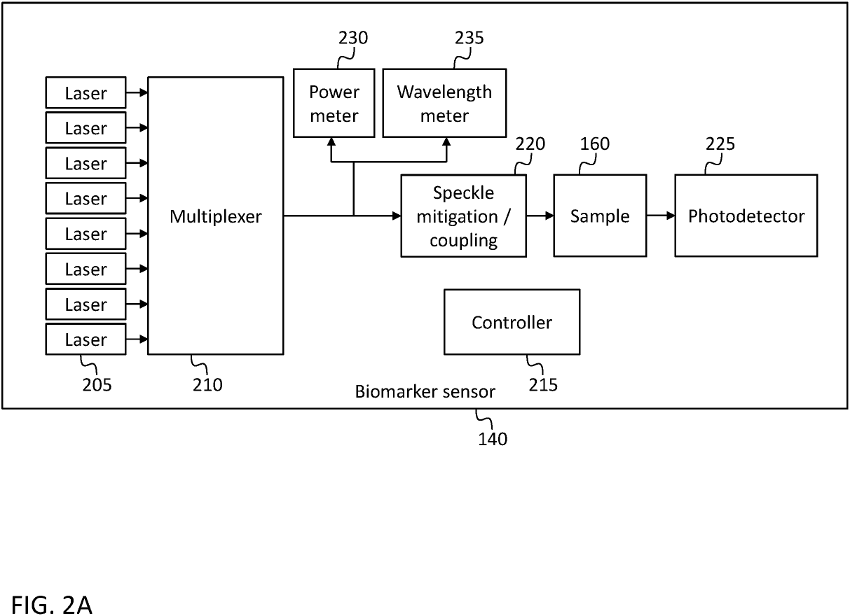

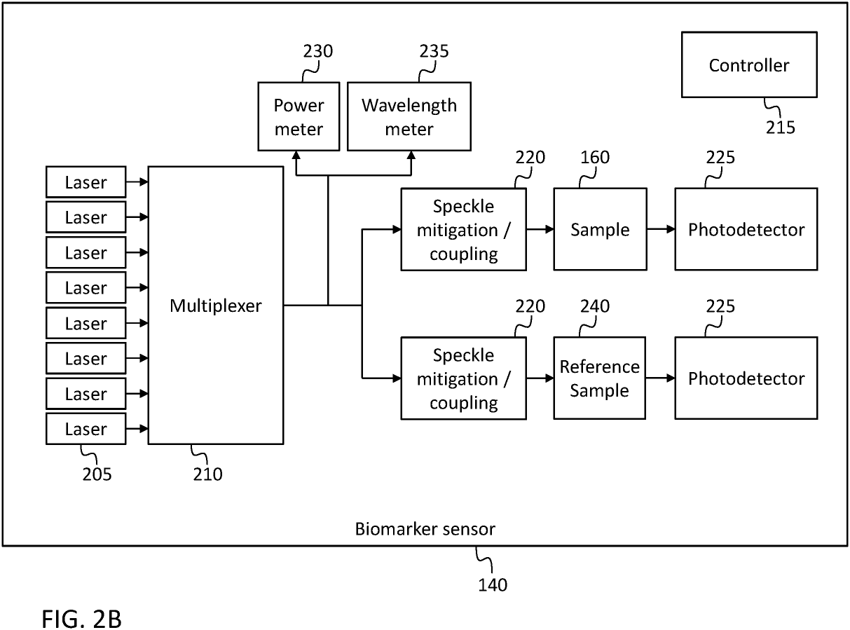

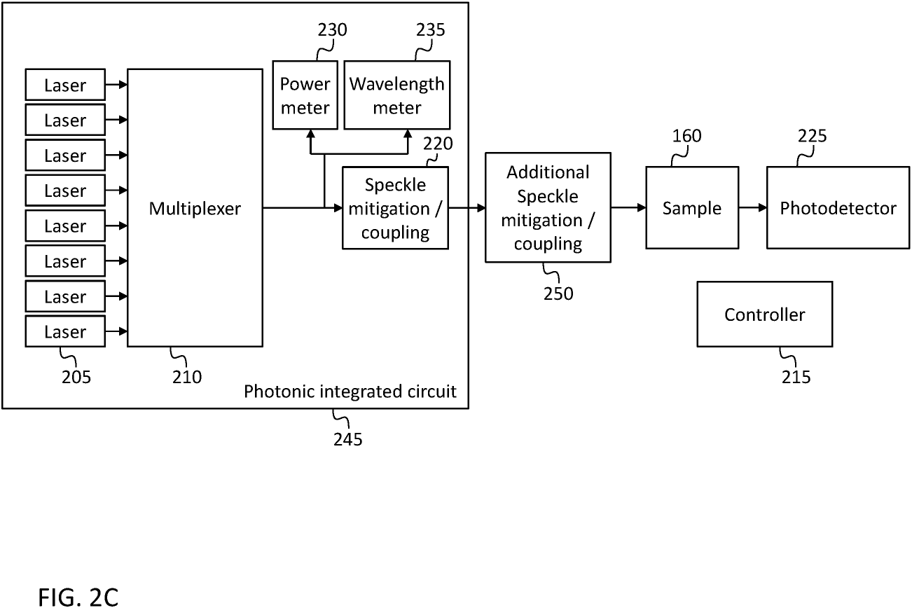

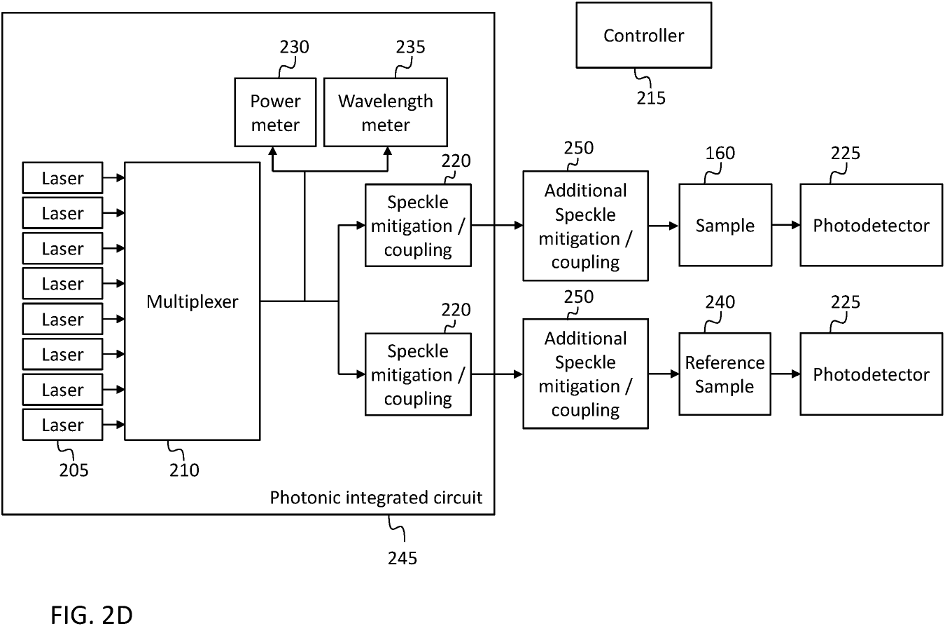

FIG. 2A is a block diagram of a biomarker sensor 140 based on a spectrophotometer, in some embodiments. Each laser 205 of an array of lasers 205 (e.g., ten or more lasers 205, not all of which are shown) is connected to a wavelength multiplexer 210 (which may be, e.g., an arrayed waveguide grating, an echelle grating, or a cascade of Mach-Zehnder interferometers). Each laser 205 operates at a different respective wavelength and is connected to an input of the wavelength multiplexer 210 corresponding to the operating wavelength. In operation, one laser is turned on at a time (e.g., by a controller 215, which may be or include a processing circuit), so that the combination of (i) the array of lasers 205 and (ii) the wavelength multiplexer 210 operates as a swept wavelength light source. In other embodiments, a different swept wavelength light source (e.g., a single widely tunable laser, or a source including an array of tunable lasers, each tunable over a different wavelength range) is used instead of the array of lasers 205 and the wavelength multiplexer 210 shown in FIG. 2A. In the embodiment of FIG. 2A, the wavelength separation between lasers 205 that are adjacent in wavelength may be between 5 nm and 50 nm, and the wavelength range may be about 2000 nm to 2500 nm (e.g., 2080 nm to 2400 nm). In some embodiments, one or more gaps may be present in the set of wavelengths (e.g., if a wavelength band within the range is of limited use because of strong absorption by water in the band).

Light from the output of the wavelength multiplexer 210 illuminates the sample holder 160. In some embodiments, a speckle mitigation system or coupling optics 220 (for reducing the spatial coherence of the probe light, and for producing a beam of the desired shape in the sample holder 160, respectively), may be present between the output of the wavelength multiplexer 210 and the sample holder 160. After interacting with the sample in the sample holder 160, the light may be detected by a photodetector 225. If the photodetector 225 is on the opposite side of the sample holder 160 from the source of the probe light (as illustrated in FIG. 2A), the probe light may be transmitted through the sample holder 160 to the photodetector 225. In other embodiments the photodetector 225 may be positioned differently, e.g., on the same side of the sample as the source of the probe light, and the probe light may reach the photodetector 225 after scattering one or more times within the sample. This type of optical path may be important for measurements made by illuminating a first location on the skin of a patient with probe light, and detecting light returning from the skin at a second location near the first location (as discussed in further detail below).

The photodiode signal may be amplified by a suitable amplifier, and converted to a digital signal by an analog to digital converter, and the resulting digital signal may be fed to the controller 215 for further processing. A power meter 230 and a wavelength meter 235 may measure the optical power and wavelength, respectively, of the probe light, and (i) corrections may be made (e.g., by the controller 215) by adjusting, e.g., the drive currents of the lasers or drive currents of heaters controlling the temperatures of respective gratings of the lasers, or (ii) errors in the transmitted power or wavelength may be compensated for when the data are analyzed. The ratio, as a function of wavelength, of (i) the optical power detected by the photodetector 225 to (ii) the optical power transmitted in the probe light may be referred to herein as a “spectrum”.

Estimates of concentrations of biomarkers may be generated, for example, by fitting a measured spectrum with a combination of signatures, each signature being the spectrum that would be expected if a single biomarker were present in the sample at a certain reference concentration.

Referring to FIG. 2B, in some embodiments, the biomarker sensor 140 includes a reference sample 240, which may be a sample holder containing a solution with known concentrations of various biomarkers. The probe light may be split as shown so that both the sample in the sample holder 160 and the reference sample 240 are illuminated and the transmitted light is measured by respective photodetectors. In such a system if the composition of the sample 160 is similar to the composition of the reference sample 240, it may be possible to generate a more accurate estimate of the composition of the fluid in the sample volume from the known composition of the reference sample 240 and the differences between the two measured spectra.

Various components, of the components illustrated in FIGS. 2A and 2B, may be fabricated on a silicon photonic integrated circuit (PIC) 245. For example, the lasers 205, wavelength multiplexer 210, the power meter 230, the wavelength meter 235, and the speckle mitigation system or coupling optics 220 may be fabricated on a photonic integrated circuit, as illustrated in FIGS. 2C and 2D. The lasers may be fabricated using III-V materials and separately heterogeneously integrated (e.g., by bonding or printing) to the photonic integrated circuit and may be in the form of distributed Bragg reflector (DBR) or distributed-feedback laser (DFB) lasers. An additional speckle mitigation arrangement or coupling optics 250 (or, in the embodiment of FIG. 2D, two additional speckle mitigation arrangements or sets of coupling optics 250) may be employed to receive the light from the optical output (or outputs) of the photonic integrated circuit 245 and to couple the light into the sample 160 (or, in the embodiment of FIG. 2D, into the sample 160 and into the reference sample 240). In such an embodiment, the spectrophotometer (and the biomarker sensor 140) may be compact, e.g., having a volume of less than 30 cubic centimeters (e.g., less than 3 cubic centimeters).

Dosing decisions may be made based on the data generated by the biomarker sensors 140. Dosing may be controlled by (i) selection of the area or permeability of the membrane of the dialyzer 120 (ii) selection of the composition of the fresh dialysate, (iii) selection of the flow rates and pressures of the blood and dialysate, and (iv) the duration of the treatment. For example, if the estimated blood urea nitrogen remains high after 4 hours of dialysis, a clinician may extend the treatment by another hour.

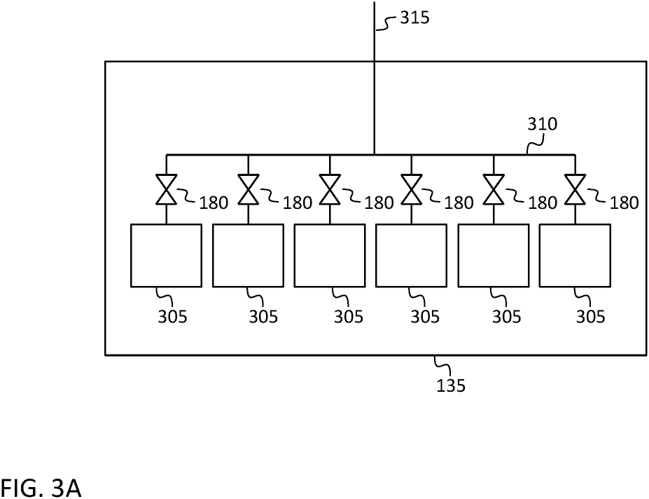

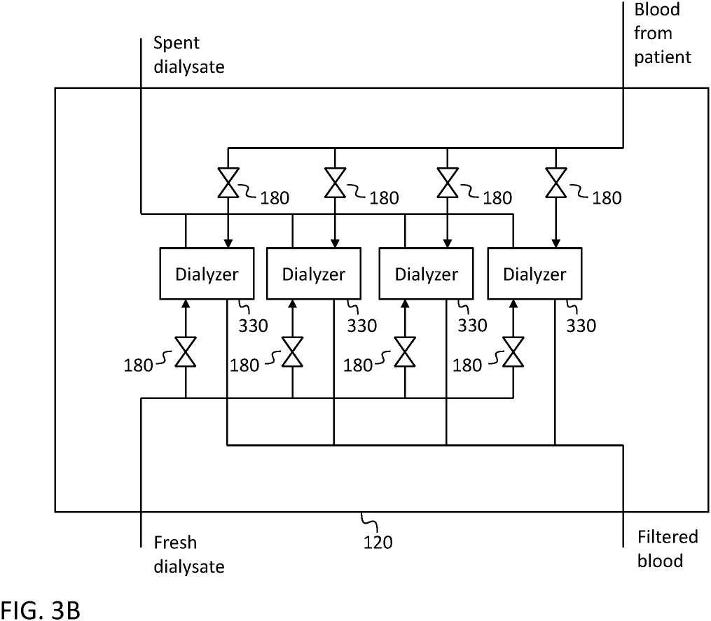

In some embodiments, dosing adjustments are made in real time, e.g., based on the data generated by the biomarker sensors 140 or based on other sensors, such as an ambulatory cuff worn by the patient. The real-time adjustments may be made, e.g., by the controller 145 of the dialysis system, e.g., by controlling one or more pumps or electrically actuated valves in the dialysis system. For example, the dialysate reservoir 135 may, as illustrated in FIG. 3A, include a number of reservoirs 305 of different dialysate solutions, each connected to a manifold 310 (which feeds the output of the dialysate reservoir 135) through a respective valve 180 controlled by the controller 145 of the dialysis system. FIG. 3A shows six reservoirs 305; some embodiments may have fewer or more reservoirs 305. For example, in an embodiment with eight reservoirs 305, one of the reservoirs 305 may contain dilute dialysate solution, and each of the other reservoirs 305 may contain a relatively concentrated solution of a respective solute (e.g., sodium (Na+), potassium (K+), calcium (Ca2+), magnesium (Mg2+), chloride (Cl-), bicarbonate, and glucose). In such an embodiment the valves 180 may be metering valves, allowing the controller 145 of the dialysis system to control the concentration of each of these solutes in the dialysate sent to the dialyzer 120.

In another example, illustrated in FIG. 3B the dialyzer 120 is a compound dialyzer including a plurality of simple dialyzers 330 connected to the blood and dialysate conduits by manifolds and valves 180 as shown. The controller 145 of the dialysis system may then adjust the effective permeability of the compound dialyzer by opening or closing valves so as to activate or idle any combination of the simple dialyzers. In the example of FIG. 3B, if each simple dialyzer 330 has twice the effective permeability of the simple dialyzer 330 to its left, then the controller 145 of the dialysis system may be able to select from among fifteen evenly spaced different effective permeabilities. In another example, the controller 145 of the dialysis system may control the pressure or flow rate of blood or dialysate by controlling the blood pump 115 and a dialysate pump.

Peritoneal dialysis uses a catheter inserted into the patient’s peritoneal space. It may be automated or manual, and the treatment may be done with one or two bags. The peritoneum is a natural membrane for water/solute equilibration. Overall, peritoneal dialysis is less physiologically stressful, with no requirement for vascular access. It is more often done at home than in a clinical environment. A biomarker sensor 140 may be used, during the removal of spent dialysate from the peritoneal space, to estimate the concentrations of various biomarkers in the spent dialysate. Such estimates may be used, for example, to guide dosing decision for subsequent dialysis sessions.

Clinical practice guidelines used to determine whether or not a dialysis session or treatment plan has been adequately carried out include several indications: the urea reduction ratio (URR), presentation of clinical symptoms, hemodynamic stability and control of blood pressure, excessive retention of fluid and overall volume control, as well as mineral metabolism. Physicians overseeing patients on dialysis may also be interested in additional biomarkers depending on the overall health status of the patient and presence of comorbidities, such as diabetes, anemia and heart disease, including heart failure.

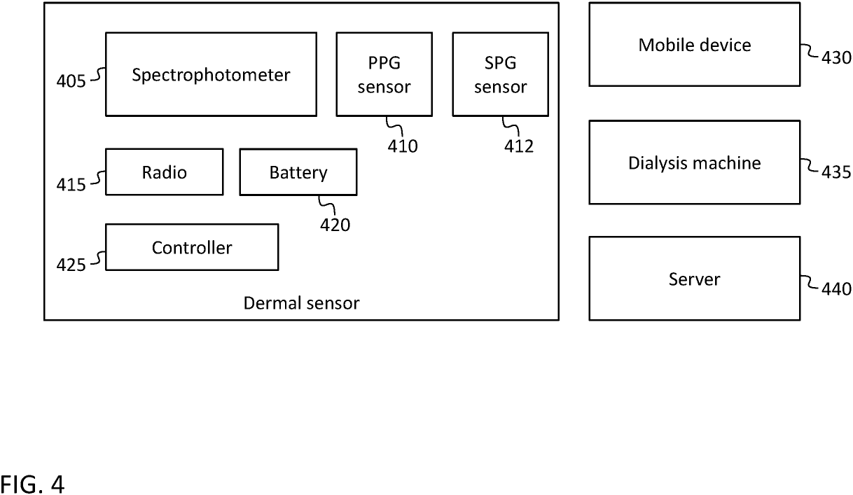

As such, an external sensing device, or “dermal sensor”, such as a wearable band or patch containing a spectrophotometer, may be an advantageous and adjunctive addition to biomarker sensors 140 fitted to a dialysis system, as discussed above. Referring to FIG. 4, the dermal sensor may include a spectrophotometer 405, a photoplethysmography (PPG) sensor 410, a speckle plethysmography (SPG) sensor 412, a radio 415 (e.g., a Bluetooth or Wi-Fi radio), a battery 420, and a controller (which may be or include a processing circuit) 425. The dermal sensor may measure spectra of the tissue of the patient (e.g., by illuminating a first location on the skin of a patient with probe light, and detecting light returning from the skin at a second location near the first location (as mentioned above). When a measurement is made in this manner, the probe light may scatter from, and propagate through, the tissue of the patient that is near the surface of the skin, and the absorption of light as it propagates through the tissue may depend on the concentrations of biomarkers in the tissue. As such, it may be possible to estimate biomarker concentrations from the spectra obtained in this manner.

The dermal sensor may transmit data based on the measurements through the radio 415, to the mobile device or application on a tablet or PC (e.g., via Bluetooth or Wi-Fi) or to the dialysis machine (either directly, e.g., via Bluetooth or Wi-Fi, or indirectly, e.g., via the mobile device 430, which may be connected to the dialysis machine 435 via Wi-Fi or through the internet, e.g., via a server 440 on the internet). During a dialysis session, a clinician may view measurements made by the dermal sensor during the current dialysis session, or measurements made (e.g., between dialysis session) before the current dialysis session.

The dermal sensor may provide various biometrics, including for example (i) biometrics that may be measured by the SPG sensor 412, such as blood pressure and blood flow, (ii) biometrics that may be measured by either the PPG sensor 410 or the SPG sensor 412, such as heart rate, heart rate variability, respiratory rate (which may be inferred from heart rate variability), and hemoglobin, (iii) biometrics that may be measured by the PPG sensor 410, such as blood oxygen saturation, and (iv) biometrics that may be measured by the spectrophotometer 405, such as body temperature, and such as the concentrations, in tissues and fluids in and under the skin of the patient, of water (a measure of total body hydration), hemoglobin, glucose, albumin, D-dimer, creatinine, urea, lactate, and toxins such as ethanol, methanol, valproate, and theophilline. The dermal sensor may be worn while a patient is undergoing dialysis treatment and may be placed in different locations on the patient based on the form factor. For example, a band may be worn on the patient’s wrist, or a patch may be placed near the fistula to help indicate the risk of thrombosis if information related to blood flow is provided. In some embodiments, a wired data connection or a wired power connection is used between the dermal sensor and the dialysis system (e.g., to power the dermal sensor or to transfer data to the dialysis system) during a dialysis session.

The adjunctive dermal sensor may provide the clinician real-time information related to the patient’s status with minimal invasiveness. It may also decrease the number of devices needed to make these assessments. Utilization of this dermal sensor may result in a decrease in the number of hemodynamic and hypotensive events and an improvement in responsiveness to patient instability during dialysis treatment. The dermal sensor may also help guide clinicians in the optimization of dialysis dosing and treatment, as well as other relevant health decisions.

The form factor of the dermal sensor may include a housing (containing, e.g., the sensors, radio, battery, and controller) secured to a band (e.g., a wrist band, chest band, arm band, leg band, or waist band), patch, or other form factor worn by the patient. The housing may be sized and dimensioned to be disposed about the body of the patient, e.g., it may have a volume of less than 200 cubic centimeters (e.g., it may have a volume of less than 10 cubic centimeters, or a volume of between 0.5 cubic centimeters and 200 cubic centimeters). The dermal sensor may be worn by the patient during dialysis treatment. Information about the patient’s biomarker status may be delivered to the clinician in real time, which may allow the clinician to make decisions related to the patient’s health and attenuate risk associated with the treatment. An example of the utility of the dermal sensor during treatment may be related to the patient’s hemodynamic stability, where monitoring the patient’s heart rate, blood pressure and hydration status may lead to improved decision-making by the clinician and improve overall treatment outcome.

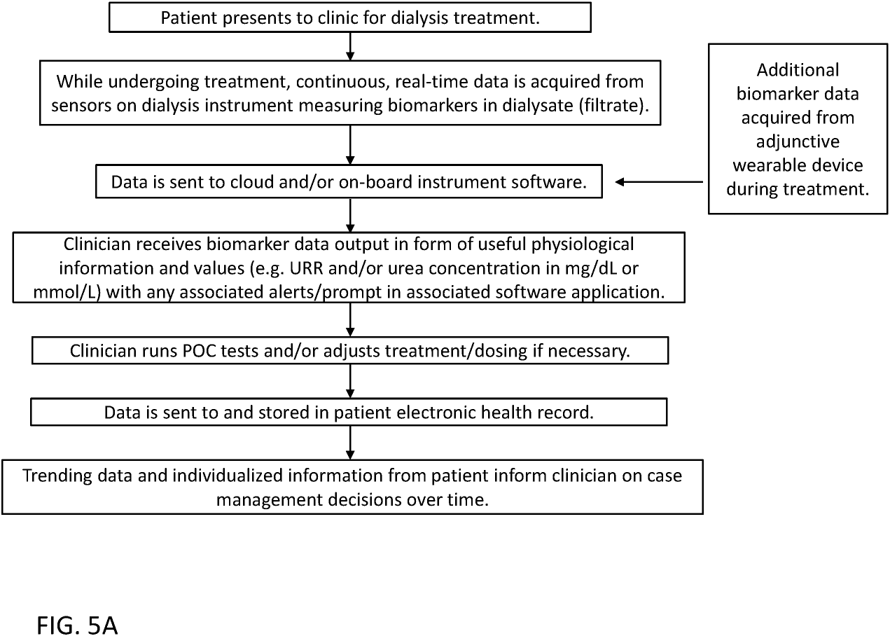

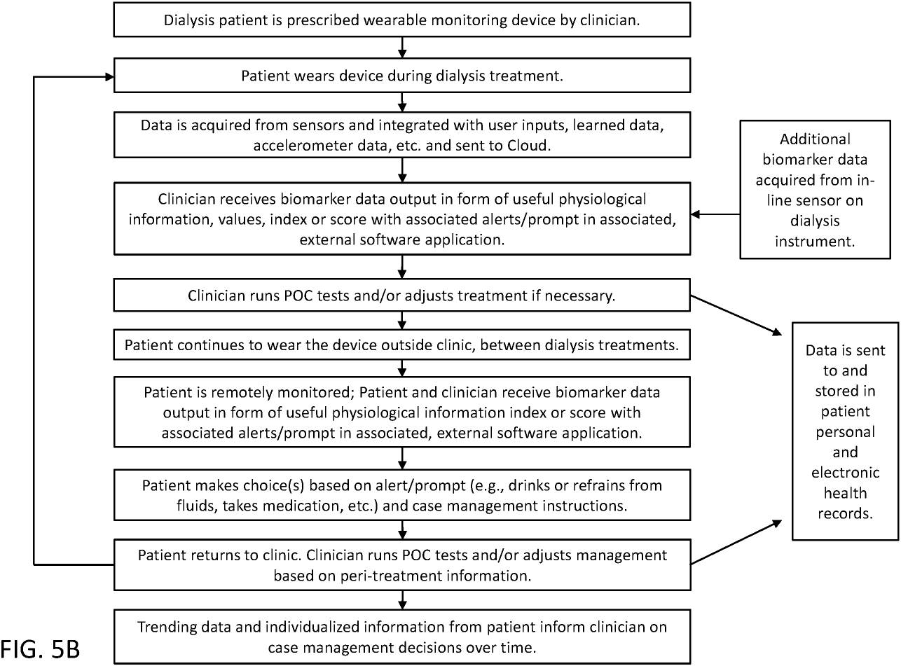

When the dermal sensor is worn during dialysis treatment, information from the dermal sensor may be combined with additional data originating from a biomarker sensor 140 on the dialysis system that is monitoring the patient’s urea concentration. The dermal sensor may also be worn by the patient before and after dialysis treatment (or not during treatment, when the patient may be at home conducting activities of daily living). Information about the patient’s health status may be collected for remote monitoring. It may be used by the patient to assist in making healthy lifestyle choices and other decisions related to the patient’s health that may reduce risk associated with kidney disease or injury, dialysis treatment, or related comorbidities. When the patient is wearing the dermal sensor, spectral data may be collected when the dermal sensor is in contact with the skin. Raw data may be pulled or pushed to an external application, and data may be collected from the device and stored on the internet (e.g., on a server 440 connected to the internet). FIGS. 5A and 5B illustrate exemplary use cases of embodiments including biomarker sensors 140 and a dermal sensor, respectively.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, when a second quantity is “within Y” of a first quantity X, it means that the second quantity is at least X-Y and the second quantity is at most X+Y. As used herein, when a second number is “within Y%” of a first number, it means that the second number is at least (1-Y/100) times the first number and the second number is at most (1+Y/100) times the first number. As used herein, the word “or” is inclusive, so that, for example, “A or B” means any one of (i) A, (ii) B, and (iii) A and B.

Each of the terms “processing circuit” and “means for processing” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed herein could be termed a second element, component, region, layer or section, without departing from the spirit and scope of the inventive concept.

Spatially relative terms, such as “beneath”, “below”, “lower”, “under”, “above”, “upper” and the like, may be used herein for ease of description to describe one element or feature’s relationship to another element(s) or feature(s) as illustrated in the figures. It will be understood that such spatially relative terms are intended to encompass different orientations of the device in use or in operation, in addition to the orientation depicted in the figures. For example, if the device in the figures is turned over, elements described as “below” or “beneath” or “under” other elements or features would then be oriented “above” the other elements or features. Thus, the example terms “below” and “under” can encompass both an orientation of above and below. The device may be otherwise oriented (e.g., rotated 90 degrees or at other orientations) and the spatially relative descriptors used herein should be interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the terms “substantially,” “about,” and similar terms are used as terms of approximation and not as terms of degree, and are intended to account for the inherent deviations in measured or calculated values that would be recognized by those of ordinary skill in the art.

As used herein, the term “major component” refers to a component that is present in a composition, polymer, or product in an amount greater than an amount of any other single component in the composition or product. In contrast, the term “primary component” refers to a component that makes up at least 50% by weight or more of the composition, polymer, or product. As used herein, the term “major portion”, when applied to a plurality of items, means at least half of the items. As used herein, any structure or layer that is described as being “made of” or “composed of” a substance should be understood (i) in some embodiments, to contain that substance as the primary component or (ii) in some embodiments, to contain that substance as the major component.

As used herein, the singular forms “a” and “an” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the inventive concept refers to “one or more embodiments of the present disclosure”. Also, the term “exemplary” is intended to refer to an example or illustration. As used herein, the terms “use,” “using,” and “used” may be considered synonymous with the terms “utilize,” “utilizing,” and “utilized,” respectively.

It will be understood that when an element or layer is referred to as being “on”, “connected to”, “coupled to”, or “adjacent to” another element or layer, it may be directly on, connected to, coupled to, or adjacent to the other element or layer, or one or more intervening elements or layers may be present. In contrast, when an element or layer is referred to as being “directly on”, “directly connected to”, “directly coupled to”, or “immediately adjacent to” another element or layer, there are no intervening elements or layers present.

Any numerical range recited herein is intended to include all sub-ranges of the same numerical precision subsumed within the recited range. For example, a range of “1.0 to 10.0” or “between 1.0 and 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Similarly, a range described as “within 35% of 10” is intended to include all subranges between (and including) the recited minimum value of 6.5 (i.e., (1 - 35/100) times 10) and the recited maximum value of 13.5 (i.e., (1 + 35/100) times 10), that is, having a minimum value equal to or greater than 6.5 and a maximum value equal to or less than 13.5, such as, for example, 7.4 to 10.6. Any maximum numerical limitation recited herein is intended to include all lower numerical limitations subsumed therein and any minimum numerical limitation recited in this specification is intended to include all higher numerical limitations subsumed therein.

Although exemplary embodiments of a system and method for monitoring during and between dialysis sessions have been specifically described and illustrated herein, many modifications and variations will be apparent to those skilled in the art. Accordingly, it is to be understood that a system and method for monitoring during and between dialysis sessions constructed according to principles of this disclosure may be embodied other than as specifically described herein. The invention is also defined in the following claims, and equivalents thereof.

What is claimed is:

1. A system, comprising:
a housing, sized and dimensioned to be disposed about the body of a patient; and
a spectrophotometer, in the housing,
the spectrophotometer being configured to illuminate the skin of the patient with probe light and to perform measurements of return light,
the return light being light returning, to a photodetector of the spectrophotometer, from the skin of the patient, as a result of the illumination by the probe light,
the spectrophotometer having an operating wavelength range including a wavelength between 2080 nm and 2400 nm.

2. The system of claim 1, wherein the spectrophotometer is configured to measure urea nitrogen.

3. The system of claim 1, wherein the housing has a volume of less than 100 cubic centimeters.

4. The system of claim 1, further comprising a radio in the housing, wherein the system is configured to transmit data, via the radio, the data being based on the measurements.

5. The system of claim 4, wherein the transmitting of the data comprises transmitting the data to a dialysis system.

6. The system of claim 5, wherein the transmitting of the data to the dialysis system comprises transmitting the data to the dialysis system wirelessly.

7. The system of claim 5, wherein the transmitting of the data to the dialysis system comprises transmitting the data during a dialysis session, the data being based on measurements performed during the dialysis session.

8. The system of claim 5, wherein the transmitting of the data to the dialysis system comprises transmitting the data during a dialysis session, the data being based on measurements performed before the dialysis session.

9. The system of claim 1, further comprising a photoplethysmography (PPG) sensor and a speckle plethysmography (SPG) sensor.

10. The system of claim 9, wherein the measurements are suitable for estimating a quantity selected from the group consisting of heart rate, blood pressure, blood flow, heart rate variability, hemoglobin, blood oxygen saturation, and respiratory rate.

11. The system of claim 1, wherein the measurements are suitable for estimating a concentration of a biomarker in tissue or fluid of the patient.

12. The system of claim 11, wherein the biomarker is selected from the group consisting of water, glucose, hemoglobin, creatinine, urea, lactate, ethanol, and albumin.

13. The system of claim 1, further comprising a band configured to secure the housing against the skin of the patient.

14. The system of claim 13, wherein the band is configured to secure the housing against the skin of the wrist of the patient.

15. The system of claim 1, further comprising an adhesive patch configured to secure the housing against the skin of the patient.

16. A method, comprising:
performing a measurement with a spectrophotometer, and
transmitting the measurement to a dialysis system,
the performing of the measurement comprising:
illuminating the skin of a patient with probe light; and
performing measurements of return light, returning, to a photodetector of the spectrophotometer, from the skin of the patient, as a result of the illumination by the probe light.

17. The method of claim 16, wherein the transmitting of the measurement to the dialysis system comprises transmitting the measurement directly to the dialysis system.

18. The method of claim 16, wherein the transmitting of the measurement to the dialysis system comprises transmitting the measurement to the dialysis system via the internet.

19. The method of claim 16, wherein the transmitting of the measurement to the dialysis system comprises transmitting the measurement during a dialysis session, the measurement being a measurement performed during the dialysis session.

20. The method of claim 16, wherein the transmitting of the measurement to the dialysis system comprises transmitting the measurement during a dialysis session, the measurement being a measurement performed before the dialysis session.
